SEQUENCE LISTING (2 sequences)

SEQ ID NO: 1 - DNA, 895 bp, Homo sapiens
gcgtcgtagtctcctgcagcgtctggggtttccgttgcagtcctcggaaccaggacctcggcgtggcctagcgagttatggcgacgaaggccgtgtgcgtgctgaagggcgacggcccagtgcagggcatcatcaatttcgagcagaaggaaagtaatggaccagtgaaggtgtggggaagcattaaaggactgactgaaggcctgcatggattccatgttcatgagtttggagataatacagcaggctgtaccagtgcaggtcctcactttaatcctctatccagaaaacacggtgggccaaaggatgaagagaggcatgttggagacttgggcaatgtgactgctgacaaagatggtgtggccgatgtgtctattgaagattctgtgatctcactctcaggagaccattgcatcattggccgcacactggtggtccatgaaaaagcagatgacttgggcaaaggtggaaatgaagaaagtacaaagacaggaaacgctggaagtcgtttggcttgtggtgtaattgggatcgcccaataaacattcccttggatgtagtctgaggccccttaactcatctgttatcctgctagctgtagaaatgtatcctgataaacattaaacactgtaatcttaaaagtgtaattgtgtgactttttcagagttgctttaaagtacctgtagtgagaaactgatttatgatcacttggaagatttgtatagttttataaaactcagttaaaatgtctgtttcaatgacctgtattttgccagacttaaatcacagatgggtattaaacttgtcagaatttctttgtcattcaagcctgtgaataaaaaccctgtatggcacttattatgaggctattaaaagaatccaaattcaaactaaa

SEQ ID NO: 2 - protein, 154 aa, Homo sapiens [sequence omitted]

(19) United States
(12) Patent Application Publication
(10) Pub. No.: US 2023/0357820 A1
(43) Pub. Date: Nov. 9, 2023

(54) TEST METHOD FOR MILD COGNITIVE IMPAIRMENT, TEST REAGENT FOR MILD COGNITIVE IMPAIRMENT, AND METHOD FOR SCREENING THERAPEUTIC CANDIDATE SUBSTANCES FOR MILD COGNITIVE IMPAIRMENT

(71) Applicant: Bioradical Research Institute Corp., Yokohama-shi, Kanagawa (JP)

(72) Inventors: Masaichi Lee, Yokohama-shi, Kanagawa (JP); Tomoko Komatsu, Yokohama-shi, Kanagawa (JP)

(73) Assignee: Bioradical Research Institute Corp., Yokohama-shi, Kanagawa (JP)

(21) Appl. No.: 18/042,053

(22) PCT Filed: Aug. 18, 2021
(86) PCT No.: PCT/JP2021/030170

§ 371 Date: Feb. 17, 2023

(30) Foreign Application Priority Data
Aug. 18, 2020 (JP) 2020-137951

(51) Int. Cl. C12Q 1/26

(52) U.S. Cl. CPC C12Q 1/26; C12Y 115/01001

(57) ABSTRACT

Provided is a test method with which mild cognitive impairment (MCI) is detectable and a measurement reagent for use in the test method. A test method for MCI according to the present invention includes a step of measuring the activity of superoxide dismutase (SOD) in a biological sample of a subject.

### TECHNICAL FIELD

The present invention relates to a test method for mild cognitive impairment, a test reagent for mild cognitive impairment, and a method for screening therapeutic candidate substances for mild cognitive impairment.

### BACKGROUND ART

Accompanying the aging of society, the number of patients with cognitive disorders such as dementia and Alzheimer’s disease or mild cognitive impairment (also referred to as mild cognitive dysfunction) in the pre-stage thereof is increasing. These cognitive impairments are diagnosed based on the diagnostic criteria for each disease and are diagnosed through a doctor’s interview, etc. (Non-Patent literature 1).

### CITATION LIST

### Non-Patent Literature

Non-Patent literature 1: The Japan Geriatrics Society, “Cognitive Function Evaluation Method and Diagnosis of Dementia” [online], The Japan Geriatrics Society, [Searched on Aug. 18, 2020], Internet, <URL: https://wwwjpn-geriat-soc.orjp/tool/tool_02.html>

### SUMMARY OF INVENTION

### Technical Problem

The progression of cognitive impairment can be slowed down through intervention in the early stage of cognitive impairment, i.e., in the stage of mild cognitive impairment (MCI). Thus, there is a demand for test methods with which MCI is detectable.

In view of this, the present invention aims to provide a test method with which MCI is detectable and a measurement reagent for use in such test method.

### Solution to Problem

In order to achieve the aims, a test method for MCI according to the present invention (also referred to as “test method” hereinafter) includes a step of measuring the activity of superoxide dismutase (SOD) in a biological sample of a subject.

A test reagent for MCI according to the present invention (also referred to as “test reagent” hereinafter) contains a reagent for measuring the activity of SOD.

A method for screening therapeutic candidate substances for MCI according to the present invention (also referred to as a “screening method” hereinafter) includes a step of selecting, as a therapeutic candidate substance for MCI, an activating substance that enhances the activity of SOD from test substances.

A detection method according to the present invention is a method for detecting the activity of SOD in a subject who has a suspected case of MCI, the method including:

a step of detecting SOD activity in a biological sample of the subject using a reagent for measuring the SOD activity.

### Advantageous Effects of Invention

According to the present invention, whether a subject has MCI is detectable using a biological sample derived from the subject.

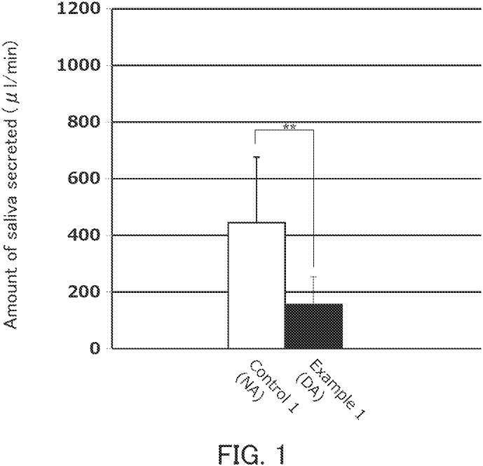

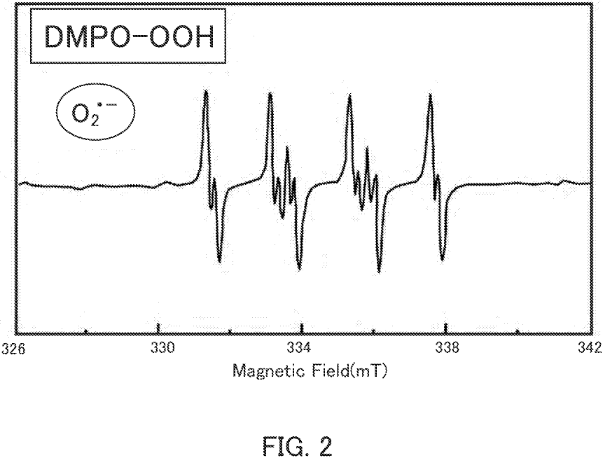

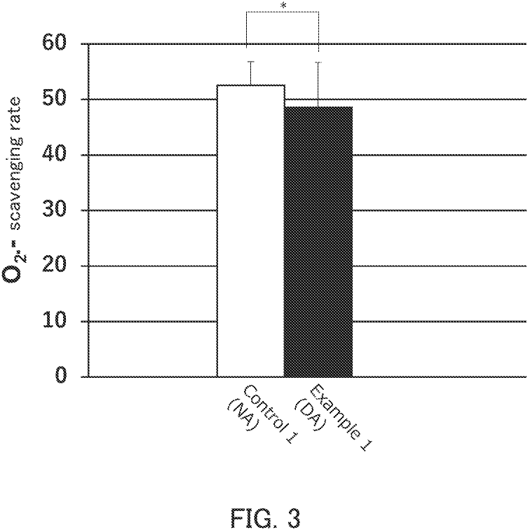

### BRIEF DESCRIPTION OF DRAWINGS

[FIG. 1] FIG. 1 is a graph showing the amount of saliva secreted by each subject in Example 1.

[FIG. 2] FIG. 2 is a diagram showing 12 absorption lines (peaks) originating from the internal magnetic field of ¹⁴N and ¹H at the β and γ positions in the Electron Spin Resonance (ESR) spectrum in Example 1.

[FIG. 3] FIG. 3 is a graph showing the ESR results for each saliva sample in Example 1.

### DESCRIPTION OF EMBODIMENTS

In the present invention, the “testing for MCI” refers to, for example, detection of MCI, determination of MCI, determination of MCI preventive effects, determination of MCI therapeutic effects, screening for MCI, determination of MCI patients who respond to a therapeutic agent, determination of therapeutic agents that take effects on individual MCI patients, test methods for diagnosing MCI, tests for treating MCI, or the like. The “determination of MCI” may be, for example, determining whether MCI occurs; testing, detection or diagnosis of MCI; determination, testing, detection, or diagnosis of the likelihood (risk) of having MCI; prediction of prognosis after treatment of MCI; or determination of the therapeutic effect of a therapeutic agent for MCI, and it may be replaced with any of these.

In the present invention, “having a disease” may refer to the state of being affected with the disease or to develop the disease.

In the present invention, the “treatment” may be used to mean any of the following: prevention of diseases; prevention, suppression, or inhibition of development of diseases; suppression, prevention, or stalling of progression of diseases or symptoms; or treatment or amelioration of diseases or symptoms, for example.

In the present invention, “mild cognitive impairment” refers to diseases classified as ICD code F06.7 (mild cognitive impairment) in the International Classification of Diseases 11th Revision (ICD11), for example. The MCI can be assessed using MoCA-J (Japanese version of MoCA), for example, and, when the score is 25 points or less, a subject can be assessed as having MCI. In the present invention, MCI may be assessed using a combination of other assessment methods, for example. Examples of the other assessment methods include Trail Making Test Japanese version (TMT-J), Clinical Dementia Rating (CDR), Functional Assessment Staging (FAST), Hasegawa’s Dementia Scale-Revised (HDS-R), Mini-Mental State Examination (MMSE), and DASC-21 (Dementia Assessment Sheet in Community-based Integrated Care System).

### MCI Marker

An MCI marker, according to the present invention, is SOD. The MCI marker, according to the present invention, is SOD, and there is no particular limitation on other constituents or conditions.

As a result of intensive studies, the inventors of the present invention found that the activity of SOD in a living organism, in particular the activity of SOD in saliva, correlates with the development of MCI, thereby establishing the present invention. According to the present invention, the likelihood of subjects having MCI (morbidity risk) and the like can be tested by measuring the activity of SOD. Also, in the present invention, because the activity of SOD serves as a target with respect to MCI, therapeutic candidate substances for MCI can be obtained through screening by using the target. Therefore, the present invention is extremely useful in the clinical and biochemical fields.

There is no particular limitation on the origin of SOD, and the origin of SOD can be set as appropriate according to the type of subjects, for example. Examples of the origin include humans and non-human animals other than humans. Examples of the non-human animals include mammals such as mice, rats, dogs, monkeys, rabbits, sheep, and horses; birds; fish; and the like. Information registered in existing databases can be referenced for SOD derived from various animals. Specific examples of a human-derived SOD include cDNA, e.g., a polynucleotide consisting of the following base sequence (Sequence ID No. 1) registered as NCBI accession number NM_000454.5, and a protein, e.g., the following amino acid sequence (Sequence ID No. 2) registered as NCBI accession number NP_000445.1. The base sequence of Sequence ID No. 1 is a sequence that encodes the amino acid sequence of Sequence ID No. 2.

Human SOD cDNA (Sequence ID No. 1)

5'-GCGTCGTAGTCTCCTGCAGCGTCTGGGGTTTCCGTTGCAGTCCTCGG
AACCAGGACCTCGGCGTGGCCTAGCGAGTTATGGCGACGAAGGCCGTGTG
CGTGCTGAAGGGCGACGGCCCAGTGCAGGGCATCATCAATTTCGAGCAGA
AGGAAAGTAATGGACCAGTGAAGGTGTGGGGAAGCATTAAAGGACTGACT
GAAGGCCTGCATGGATTCCATGTTCATGAGTTTGGAGATAATACAGCAGG
CTGTACCAGTGCAGGTCCTCACTTTAATCCTCTATCCAGAAAACACGGTG
GGCCAAAGGATGAAGAGAGGCATGTTGGAGACTTGGGCAATGTGACTGCT
GACAAAGATGGTGTGGCCGATGTGTCTATTGAAGATTCTGTGATCTCACT
CTCAGGAGACCATTGCATCATTGGCCGCACACTGGTGGTCCATGAAAAAG
CAGATGACTTGGGCAAAGGTGGAAATGAAGAAAGTACAAAGACAGGAAAC
GCTGGAAGTCGTTTGGCTTGTGGTGTAATTGGGATCGCCCAATAAACATT
CCCTTGGATGTAGTCTGAGGCCCCTTAACTCATCTGTTATCCTGCTAGCT
GTAGAAATGTATCCTGATAAACATTAAACACTGTAATCTTAAAAGTGTAA
TTGTGTGACTTTTTCAGAGTTGCTTTAAAGTACCTGTAGTGAGAAACTGA
TTTATGATCACTTGGAAGATTTGTATAGTTTTATAAAACTCAGTTAAAAT
GTCTGTTTCAATGACCTGTATTTTGCCAGACTTAAATCACAGATGGGTAT
TAAACTTGTCAGAATTTCTTTGTCATTCAAGCCTGTGAATAAAAACCCTG
TATGGCACTTATTATGAGGCTATTAAAAGAATCCAAATTCAAACTAAA-3
'

Human SOD protein (Sequence ID No. 2)

MATKAVCVLKGDGPVQGIINFEQKESNGPVKVWGSIKGLTEGLHGFHVHE
FGDNTAGCTSAGPHFNPLSRKHGGPKDEERHVGDLGNVTADKDGVADVSI
EDSVISLSGDHCIIGRTLVVHEKADDLGKGGNEESTKTGNAGSRLACGVI
GIAQ

SOD can be used as an MCI marker and can be particularly suitably used as a marker for MCI resulting from Down syndrome, a marker for MCI resulting from amyotrophic lateral sclerosis (ALS), and a marker for MCI resulting from SOD degeneration.

The marker according to the present invention may be the activity of SOD protein, more specifically the superoxide anion (superoxide, O₂·⁻) scavenging ability of SOD protein.

As will be described later, the marker according to the present invention can also be used as a marker for diagnosing or detecting MCI, a marker for predicting the prognosis of MCI, or a marker for predicting or determining therapeutic effects on MCI.

### Test Method for MCI

As described above, a test method for mild cognitive impairment (MCI) according to the present invention includes a step of measuring the activity of superoxide dismutase (SOD) in a biological sample of a subject. The test method according to the present invention includes measuring the activity of SOD in the biological sample of the subject as the MCI marker, and there is no particular limitation on other steps and conditions. The description of MCI and the marker of the present invention can be applied to the test method of the present invention.

With the test method of the present invention, it is possible to perform testing for MCI, for example. The “testing for MCI” refers to detection of MCI, determination of MCI, determination of MCI preventive effects, determination of MCI therapeutic effects, determination of MCI patients who respond to a therapeutic agent, determination of therapeutic agents that take effects on individual MCI patients, test methods for diagnosing MCI, tests for treating MCI, or the like. The “determination of MCI” may be, for example, determining whether MCI occurs; testing, detection or diagnosis of MCI; determination, testing, detection, or diagnosis of the likelihood (risk) of having MCI; prediction of prognosis after treatment of MCI;, or determination of the therapeutic effect of a therapeutic agent for MCI.

In the test method according to the present invention, examples of the subject include humans and non-human animals other than humans. As described above, examples of the non-human animals include mammals such as mice, rats, dogs, monkeys, rabbits, sheep, and horses; birds; fish; and the like.

In the test method according to the present invention, there is no particular limitation on the type of the biological sample, and examples thereof include body fluids, body fluid-derived cells, organs, tissues, and cells that are isolated from living organisms. Examples of the body fluid include blood; saliva; urine; lymphatic fluid; synovial fluid of joints; cerebrospinal fluids such as bone marrow aspirate and cerebrospinal fluid; and the like. Specific examples of the blood include whole blood, serum, and plasma. Examples of the body fluid-derived cells include blood-derived cells, and specific examples thereof include blood cells such as blood cells, leukocytes, and lymphocytes. Further, with the MCI marker of the present invention, MCI of a subject can be tested by the activity of SOD in saliva, for example. Therefore, the biological sample is preferably saliva because it is possible to reduce the burden on patients and doctors, for example.

Examples of the activity of SOD to be measured include the superoxide (O₂·⁻) scavenging ability of SOD, for example. The activity of SOD may be measured by measuring the O₂·⁻ scavenging ability of SOD for the biological sample, or it may be measured by measuring the O₂·⁻ scavenging ability of SOD for SOD obtained from the biological sample after extraction, rough purification, or purification, for example. There is no particular limitation on the method for measuring the O₂·⁻ scavenging ability of SOD, and a known method can be adopted. Specific examples of the method for measuring the O₂·⁻ scavenging ability of SOD include the Electron Spin Resonance (ESR) spin-trapping method using xanthine and xanthine oxidase as O₂·⁻ generators and a spin-trapping agent; spectrophotometry using tetrazolium salts (color formers) such as nitro blue tetrazolium (NBT) and 2,3,5-triphenyltetrazolium chloride (XTT); a chemiluminescent method using a chemiluminescent probe such as Cypridina luciferin analog (MCLA) and lucigenin; a method utilizing reduction of cytochrome C; a method utilizing reduction of tetranitromethane (TNM); a method utilizing oxidation (chain reaction) of epinephrine (adrenaline); a method utilizing oxidation (chain reaction) of lactate dehydrogenase-amyotrophic lateral sclerosis(NADH); and a measurement method through the formation of a lactoperoxidase/superoxide complex, furthermore the ESR spin-trapping method is preferable because it is possible to suppress the effects of other reactive oxygen species such as hydrogen peroxide, hydroxyl radicals, and singlet oxygen and to specifically detect O₂·⁻, and the ESR spin-trapping method has good sensitivity. In the measurement method, qualitative measurement (analysis) may be performed, or quantitative measurement (analysis) may be performed. In the latter case, the activity of SOD can also be referred to as an SOD activity value.

The test method according to the present invention further includes a step of testing the likelihood of the subject having MCI by comparing the SOD active value in the biological sample of the subject (also referred to as a “test biological sample” hereinafter) with a reference value, for example. There is no particular limitation on the reference value, and examples thereof include the SOD activity values in healthy subjects, MCI patients, and MCI patients at respective levels of cognitive impairment. In the case of prognostic assessment, the reference value may be the SOD activity value obtained before or after treating the same subject (e.g., immediately after treatment), for example.

The reference value can be obtained using biological samples isolated from healthy subjects and/or MCI patients (also referred to as a “reference biological sample” hereinafter) as described above, for example. Specifically, reference biological samples are isolated from multiple healthy subjects and multiple MCI patients, the SOD activity thereof is measured, and then the reference value can be set, based on the obtained SOD activity, to a value that maximizes the sensitivity and specificity of the test for the likelihood of MCI. Also, in the case of prognostic assessment, a reference biological sample isolated from the same subject after treatment may be used, for example. The reference value may be measured at the same time as the measurement for the test biological sample from the subject or may be measured in advance, for example. The latter case is preferable because there is no need to obtain a reference value each time a test biological sample from the subject is measured, for example. It is preferable that the test biological sample from the subject and the reference biological sample are collected under the same conditions, and the activities of SOD therein are measured under the same conditions, for example.

There is no particular limitation on the method for assessing the likelihood of a subject having MCI in the step of testing, and the assessment method can be determined as appropriate according to the type of the reference value. As specific examples, when the SOD activity value in the test biological sample from the subject is the same as the SOD activity value in the reference biological samples of the healthy subjects (when there is no significant difference), when the SOD activity value in the test biological sample from the subject is significantly higher than the SOD activity value in the reference biological samples of the healthy subjects, and/or when the SOD activity value in the test biological sample from the subject is significantly higher than the SOD activity value in the reference biological samples of the MCI patients, the subject is assessed as having no or little likelihood (also referred to as “risk” or “degree of risk,” and the same applies to the following) of having MCI. Further, when the SOD activity value in the test biological sample from the subject is significantly lower than the SOD activity value in the reference biological samples of the healthy subjects, when the SOD activity value in the test biological sample from the subject is the same as the SOD activity value in the reference biological samples of the MCI patients (when there is no significant difference), and/or when the SOD activity value in the test biological sample from the subject is significantly lower than the SOD activity value in the reference biological samples of the MCI patients, the subject can be assessed as likely, or highly likely (having high risk), to have MCI. Also, in the step of testing, the level of MCI can be assessed by comparing the SOD activity value in the test biological sample from the subject with the SOD activity values in the reference biological samples of the MCI patients at respective levels of cognitive impairment. Specifically, when the test biological sample from the subject has an SOD activity value similar to that in the reference biological samples at a certain level of cognitive impairment (when there is no significant difference), for example, the subject can be assessed as likely to have cognitive impairment at that level.

When the prognostic state is assessed in the step of testing, an assessment may be made in a manner similar to the above-described manner or can be assessed using the SOD activity value in a reference biological sample obtained after treating the same subject, for example. As specific examples, when the SOD activity value in the test biological sample from the subject is the same as the reference value (when there is no significant difference), and/or when the SOD activity value in the test biological sample from the subject is significantly lower than the reference value, the subject can be assessed as likely to relapse or worsen after the treatment. Also, when the SOD activity value in the test biological sample from the subject is significantly higher than the reference value, the subject can be assessed as having no or little likelihood of a relapse of MCI after the treatment.

In the present invention, in the step of testing, biological samples may be collected from the same subject over time, and the SOD activity values in the biological samples may be compared with each other, for example. As a result, in the step of testing, when the activity value decreases over time, it can be determined that the likelihood of having MCI has increased, and, when the expression level increases over time, it can be determined that the likelihood of having MCI has decreased or the patient has been cured, for example.

In the test method according to the present invention, treatment may be administered to the subject based on the results of the step of testing, for example. Specifically, the test method according to the present invention may include a step of administering a therapeutic agent for MCI to a subject who has received a test result indicating that the subject has MCI, for example. Conditions such as an administration form, an administration period, a dosage, and an administration interval of the therapeutic agent for MCI and the like can be set as appropriate according to the type of the therapeutic agent for MCI. The therapeutic agent for MCI may be a therapeutic candidate substance obtained through a later-described screening method according to the present invention.

As described above, the test method according to the present invention can be used as a method for diagnosing or detecting MCI, a method for predicting the prognosis of MCI, and a method for predicting or determining effects of MCI treatment, for example. Therefore, the test method according to the present invention can also be used as a companion diagnostic method for selecting patients (responders) who respond to the therapeutic agent for MCI and adjusting the dosage of the therapeutic agent for MCI.

### Test Reagent

As described above, the test reagent according to the present invention contains a reagent for measuring the activity of superoxide dismutase (SOD). The test reagent according to the present invention contains a reagent for measuring the SOD activity, and there is no particular limitation on other constituents and conditions. With the test reagent of the present invention, the test method according to the present invention can be easily performed. The description of the mild cognitive impairment (MCI) marker and the test method of the present invention can be applied to the test reagent of the present invention.

The reagent for measuring the SOD activity can be determined as appropriate according to the method for measuring the SOD activity, for example. When the SOD activity is to be measured using the Electron Spin Resonance (ESR) method, specific examples of the reagent for measuring the SOD activity include a superoxide (O₂·⁻) generator and a spin-trapping agent. Examples of the O₂·⁻ generator include a combination of xanthine and xanthine oxidase. Examples of the spin-trapping agent include 5,5-dimethyl-1-pyrroline-N-oxide (DMPO), 5-(diethoxyphophoryl)-5-methyl-1-pyrroline-N-oxide (DEPMPO), and 5-(2,2-dimethyl-1,3-propoxycyclophosphoryl)-5-methyl-1-pyrroline N-oxide (CYPMPO).

As described above, with the test reagent according to the present invention, the test method according to the present invention can be easily performed. Therefore, the test reagent of the present invention is preferably used in the test method according to the present invention.

It can also be said that the present invention relates to use of a reagent for measuring the SOD activity in order to test the likelihood of MCI, for example.

In the present invention, reagents may be mixed and provided, or some or all of the reagents may be provided separately. In the latter case, the test reagent according to the present invention can also be referred to as a test kit.

The test reagent according to the present invention may contain other constituents, for example. Examples of the other constituents include a pretreatment reagent for biological samples, instructions for use, and the like.

The test reagent according to the present invention may contain a reagent for measuring SOD, instead of the reagent for measuring the SOD activity. The reagent for measuring SOD may be a reagent for measuring SOD protein, or a reagent for measuring mRNA that encodes SOD protein, for example. It is possible to use an antibody against SOD or the like as the reagent for measuring SOD protein, for example. Examples of the reagent for measuring mRNA that encodes the SOD protein include reverse transcriptase, dNTPs, polymerases, and primers.

### Method and Reagent for Diagnosing MCI

A method for diagnosing mild cognitive impairment (MCI) according to the present invention includes a step of measuring the activity of superoxide dismutase (SOD) in a biological sample of a subject. Also, a reagent for diagnosing MCI according to the present invention contains a reagent for measuring the expression of SOD. Note that the description of the test method and the test reagent of the present invention can be applied thereto.

### Screening Method

As described above, a method for screening therapeutic candidate substances for mild cognitive impairment (MCI) according to the present invention includes a step of selecting, as a therapeutic candidate substance for MCI, an activating substance that enhances the activity of superoxide dismutase (SOD) from test substances. In the present invention, the target of therapeutic candidate substances for MCI is the SOD activity, and there is no particular limitation on other steps and conditions. The description of the MCI marker, the test method, and the test reagent of the present invention can be applied to the screening method of the present invention.

Examples of the test substance include low-molecular-weight compounds, peptides, proteins, and nucleic acids. Examples of the low-molecular-weight compounds include libraries of known low-molecular-weight compounds. Examples of the peptides include linear, branched, or cyclic peptides, and amino acids that constitute a peptide are natural amino acids, modified amino acids, artificial amino acids, or a combination thereof. The protein may be either a native protein or an artificial protein. Examples of the protein include antibodies, growth factors, proliferators, and variants thereof. Examples of the nucleic acids include substances that suppress the expression of SOD, and specific examples thereof include substances that suppress transcription of mRNA from the SOD gene, substances that cleave the transcribed mRNA, and substances that suppress protein translation from mRNA. Examples of the nuclei acids include RNA interference agents such as siRNA, antisense, and ribozymes.

The step of selecting includes, for example, a step of causing the test substance to be also present in a mixture containing superoxide and SOD and measuring the SOD activity, and a step of choosing the test substance as the therapeutic candidate substance when the SOD activity obtained in the step of causing the test substance to be also present in a mixture containing superoxide and SOD and measuring the SOD activity is higher than that in a control mixture in which the test substance is not present. In the step of causing the test substance to be also present in a mixture containing superoxide and SOD and measuring the SOD activity, the description of the above-described method for measuring the SOD activity value can be applied to the measurement of the SOD activity.

### Detection Method

A detection method according to the present invention is a method for detecting the activity of superoxide dismutase (SOD) in a subject who has a suspected case of mild cognitive impairment (MCI), the detection method including a step of detecting the SOD activity in a biological sample of the subject by using a reagent for measuring the SOD activity. The detection method according to the present invention includes detecting the activity of SOD in the biological sample of the subject, and there is no particular limitation on other steps and conditions. The description of the MCI marker, the test method, and the test reagent of the present invention can be applied to the detection method of the present invention.

The subject who has a suspected case of MCI may be, for example, a person who is subjectively suspected of having MCI, or a person who has been determined to have a suspected case of MCI, or possibly have MCI, as a result of a medical examination by a doctor or the like. Examples of the persons who are subjectively suspected of having MCI include persons who have some subjective symptoms and persons who wish to undergo preventive examinations. Examples

The following describes examples of the present invention. However, the present invention is not limited to the following examples. Commercially available reagents were used based on protocols thereof, unless otherwise stated.

### Example 1

Down syndrome patients were used as mild cognitive impairment (MCI) models, and it was confirmed that the superoxide dismutase (SOD) activity decreased in saliva derived from the Down syndrome patients.

### Subject

It is said that Down syndrome patients show MCI in an early stage. In view of this, whether the SOD activity correlates with MCI was examined using Down syndrome patients as MCI models.

A Down syndrome patient group (DA, Example 1, n=31, 22 males, nine females, average age 48.9±6.5 years), and a healthy subject group (NA, Control 1, n=24, seven males, 14 females, average age 47.1±4.9 years) were used as subjects. Each subject provided written informed consent. Also, when selecting subjects, persons who had used a corticosteroid or immunosuppressant agent for a long period, persons who had a history of use of antibiotics within the past three months, and persons who had been treated with an antifungal agent within the past six weeks were excluded.

### Collection of Saliva

Saliva was collected using a commercially available saliva collection tube (Salikids (registered trademark) manufactured by Sarstedt K.K.), as follows. First, each subject was allowed to introduce a cotton roll attached to Salikids (registered trademark) into the oral cavity for 5 minutes so that the cotton roll sufficiently absorbed the saliva of the subject. Then, the cotton roll impregnated with saliva was inserted into a suspending insert of Salikids (registered trademark), a cap was closed, and then centrifugation was performed under the conditions of 3000×g and 3 minutes. After separation was performed, the supernatant fraction in the tube was used as a saliva sample of each subject and stored at -40° C. Note that, when saliva was collected, each subject was prohibited from eating, drinking, taking drugs orally, brushing teeth, and Professional Mechanical Tooth Cleaning (PMTC) for 1 hour before the collection thereof.

### Amount of Saliva Secreted

The amounts of saliva secreted by the subjects are shown in FIG. 1. FIG. 1 is a graph showing the average of the amounts of saliva secreted by subjects in each group, and the average of the amounts of secreted saliva in each group is shown on the vertical axis, and the subject group is shown on the horizontal axis. As shown in FIG. 1, the Down syndrome patient group (Example 1) exhibited a significant decrease in the amount of secreted saliva as compared to the healthy subject group (Control 1). Note that the asterisk in FIG. 1 indicates a p-value, and ** means p<0.01. Also, statistical analysis was performed using the Student-Newman-Keuls method (the same applies to the following).

### Measurement of Superoxide (O₂·_{¯}) Scavenging Ability of Saliva

The superoxide (O₂·_{¯}) scavenging ability of each saliva sample was checked using the Electron Spin Resonance (ESR) spin-trapping method using 5,5-dimethyl-1-pyrroline-N-oxide (DMPO) as a spin-trapping agent. The O₂·⁻ production system was a xanthine/xanthine oxidase (XO) production system. Specifically, 20 µ1 of 0.1 U/ml xanthine oxidase was added to 180 µ1 of 0.1 mol/l phosphate buffered saline (pH 7.2) containing 20 µ1 of 440 mmol/l DMPO and 20 µ1 of 362 µmol/l xanthine so as to generate O₂·⁻. Then, the resulting mixture (200 µ1) was transferred to a flat cell, and the generation of O₂·⁻ was checked by measuring the DMPO-OH spin adduct using the X-band ESR spin-trapping method. ESR measurement conditions will be described later.

As indicated by Formula (A) below, the generated O₂·⁻ reacts with DMPO added to the reaction system to generate nitroxide (DMPO-OOH), which is a stable radical detectable by ESR. As shown in FIG. 2, this radical exhibits 12 absorption lines (peaks) derived from the internal magnetic field of ¹⁴N and ¹H at the β and γ positions in the ESR spectrum.

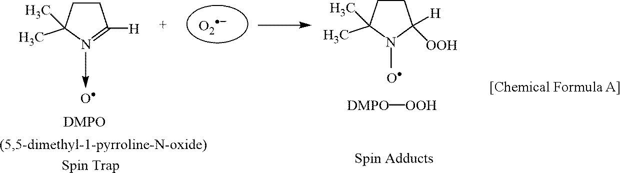

When a saliva sample is added to the reaction system, the intensity of signals obtained through ESR changes. The O₂·⁻ scavenging ability of the saliva sample was measured as follows. First, 20 µ1 of 0.1 U/ml XO was added to 180 µ1 of 0.1 mol/1 phosphate buffered saline (pH 7.2) containing 20 µ1 of 440 mmol/l DMPO, 20 µ1 of 362 µmol/1 xanthine, and 20 µ1 of the saliva sample so as to generate O₂·⁻.Then, the obtained mixture (200 µ1) was transferred to a flat cell, and the O₂·⁻ scavenging ability of the saliva sample was determined by measuring the DMPO-OH spin adduct using the X-band ESR spin-trapping method.

In the ESR measurement, an electron spin resonance device (JES-RE 3X, X-band spectrometer, manufactured by JEOL Ltd.) was connected to WIN-RAD ESR data analyzer (Radical Research, Tokyo, Japan), and the following measurement conditions were used. The hyperfine coupling constant was calculated using the resonance frequency measured using a microwave frequency counter, and the resonance electric field was measured using an electric field measuring device ES-FC5 (JEOL Ltd.). The detected spin adducts were quantified from the ESR spectra of manganese oxide standards. The actually measured signal intensity was expressed by a relative height normalized to the signal intensity of the ESR spectra of the manganese oxide standards. Also, the average for each group (Example 1 and Control 1) was calculated, and the O₂·⁻ scavenging ability was calculated as a relative value obtained using the reaction system to which a saliva sample was not added as the standard (100%). The results therefor are listed in FIG. 3.

### ESR Measurement Conditions

Device:

Electron Spin Resonance (ESR) device (JES-RE 3X, X-band spectrometer, manufactured by JEOL Ltd.)

Measurement conditions:
Microwave power: 8.00 mW
Sweep time: 1 minute
Sweep width: 334.8±5 mT
Magnetic field modulation: 100 kHz 0.079 mT
Gain: ×400
Sweep time: 1 minute
Time constant: 0.03 seconds

FIG. 3 is a graph showing the ESR results for each saliva sample. In FIG. 3, the relative value of O₂·⁻ scavenging rate is shown on the vertical axis, and the type of saliva sample is shown on the horizontal axis. As shown in FIG. 3, the Down syndrome patient group (Example 1) had a significantly lower O₂·⁻ scavenging ability (SOD activity) as compared to the healthy subject group (Control 1).

Based on the above, it was found that the SOD activity decreased in the saliva of Down syndrome patients.

Because chromosome 21 is a trisomy in Down syndrome, it has been said that the SOD activity value increases. However, as shown in Example 1, it was found that the SOD activity decreased, as compared to the healthy subjects. Thus, in the Down syndrome patients, the expression level of SOD increased, and the apparent SOD activity value increased due to the trisomy of chromosome 21, but actually it is presumed that SOD in Down syndrome patients is denatured into SOD with weakened superoxide (O₂·⁻) scavenging ability. Note that the present invention is in no way limited to this presumption.

Further, it is known that Down syndrome patients are susceptible to dementia due to premature aging, and to exhibit mild cognitive impairment (MCI) symptoms. Therefore, it is conceivable that measurement of the SOD activity is also useful for diagnosis of MCI.

### Example 2

It was confirmed that the superoxide dismutase (SOD) activity decreased in the saliva derived from mild cognitive impairment (MCI) patients.

Cognitive function test (Japanese version of MoCA: MoCA-J) was performed on 22 healthy subjects (21 to 68 years old) who gave informed consent in writing, and cognitive functions (visuospatial, executive function, nomenclature, memory, attention, repetition, word recall, abstraction, delayed recall, and orientation) were measured. Subjects with a MoCA-J score of 25 points or less were evaluated as MCI patients (Example 2, n=8, average cognitive function 22.5), and subjects with a MoCA-J score of 26 points or more were evaluated as healthy subjects (Control 2, n=14, average cognitive function 27.9). When selecting subjects, persons who had used a corticosteroid or immunosuppressant agent for a long period, persons who had a history of use of antibiotics within the past three months, and persons who had been treated with an antifungal agent within the past six weeks were excluded.

Then, saliva was collected, and the superoxide (O₂·⁻) scavenging ability (SOD activity) was measured in a manner similar to that in Example 1, except that the subjects of Example 2 (Example 2 and Control 2) were used instead of the subjects of Example 1 (Example 1 and Control 1). Four to six saliva samples were collected from each subject, and the O₂·⁻ scavenging ability of the multiple samples was measured, the average was calculated, and the calculated average was used as the O₂·⁻ scavenging ability of each subject.

The results therefor are listed in FIG. 1. As listed in Table 1 below, the saliva of Example 2 had less than half the SOD activity as compared to the saliva of Control 2.

TABLE 1

| Subject No. | Cognitive function test results (MCI) | O₂·⁻ suppression ratio | Average cognitive function | Average suppression ratio |
|---|---|---|---|---|
| 23 | 20 | 16.37% | 22.5 (MCI group/Ex. 2) | 10.9% |
| 17 | 21 | 11.08% | | |
| 8 | 22 | 21.74% | | |
| 10 | 22 | 13.39% | | |
| 9 | 23 | 2.36% | | |
| 13 | 23 | 17.55% | | |
| 1 | 24 | 3.46% | | |
| 5 | 25 | 1.22% | | |
| 6 | 26 | 28.67% | 27.9 (Healthy subject group/Control 2) | 21.8% |
| 16 | 26 | 33.01% | | |
| 3 | 27 | 8.57% | | |
| 14 | 27 | 50.04% | | |
| 21 | 27 | 16.52% | | |
| 22 | 27 | 28.77% | | |
| 7 | 28 | 17.34% | | |
| 15 | 28 | 11.08% | | |
| 2 | 29 | 8.43% | | |
| 11 | 29 | 21.16% | | |
| 12 | 29 | 3.97% | | |
| 18 | 29 | 10.05% | | |
| 19 | 29 | 7.98% | | |
| 20 | 29 | 59.83% | | |

Based on the above, it was found that, because the SOD activity decreased in the saliva of MCI patients, the SOD activity functions as a marker for MCI.

Although the present invention was described with reference to embodiments and examples, the present invention is not limited to the above-described embodiments and examples. Various changes that can be understood by those skilled in the art can be made to the constituents and details of the present invention within the scope of the present invention.

This application is based upon and claims the benefit of priority from Japanese Patent Application No. 2020-137951, filed on Aug. 18, 2020, and the entire content of the disclosure is incorporated herein by reference.

### Supplementary Descriptions

Some or all of the above embodiments and examples may be described as, but not limited to, the following supplementary descriptions.

### (Supplementary Description 1)

A marker for detecting mild cognitive impairment (MCI), the marker being superoxide dismutase (SOD).

### (Supplementary Description 2)

The marker according to Supplementary Description 1, wherein the marker is activity of superoxide dismutase (SOD).

### (Supplementary Description 3)

The marker according to Supplementary Description 1 or 2, wherein the marker is superoxide dismutase (SOD) in saliva.

### (Supplementary Description 4)

A test method for mild cognitive impairment (MCI), the method including a step of measuring activity of superoxide dismutase (SOD) in a biological sample of a subject.

### (Supplementary Description 5)

The test method according to Supplementary Description 4, wherein an SOD activity value is measured in the step of measuring activity of superoxide dismutase (SOD) in a biological sample of a subject.

### (Supplementary Description 6)

The test method according to Supplementary Description 5, further includes a step of testing the likelihood of the subject having mild cognitive impairment (MCI) by comparing the SOD activity value in the biological sample of the subject with a reference value,

wherein the reference value is an SOD activity value in a biological sample of a healthy subject or an SOD activity value in a biological sample of an MCI patient.

### (Supplementary Description 7)

The test method according to Supplementary Description 6, wherein, in the step of testing, when the superoxide dismutase (SOD) activity value in the biological sample of the subject is lower than the SOD activity value in the biological sample of the healthy subject, when the SOD activity value in the biological sample of the subject is the same as the SOD activity value in the biological sample of the mild cognitive impairment (MCI) patient, or when the SOD activity value in the biological sample of the subject is lower than the SOD activity value in the biological sample of the MCI patient, the subject is assessed as likely to have MCI.

### (Supplementary Description 8)

The test method according to any one of Supplementary Descriptions 4 to 7, wherein the SOD activity is superoxide scavenging activity.

### (Supplementary Description 9)

The test method according to any one of Supplementary Descriptions 4 to 8, wherein the biological sample is saliva.

### (Supplementary Description 10)

The test method according to any one of Supplementary Descriptions 4 to 9, wherein the testing for mild cognitive impairment (MCI) is detection of MCI, determination of MCI, determination of an MCI preventive effect, determination of an MCI therapeutic effect, screening for MCI, determination of an MCI patient who responds to a therapeutic agent, determination of a therapeutic agent that takes an effect on an individual MCI patient, a test method for diagnosing MCI, or a test for treating MCI.

### (Supplementary Description 11)

The test method according to any one of Supplementary Descriptions 4 to 10, the test method further including a step of administering a therapeutic agent for mild cognitive impairment (MCI) to a subject who has received a test result indicating that the subject has MCI.

### (Supplementary Description 12)

A test reagent for mild cognitive impairment (MCI), the test reagent containing a reagent for measuring activity of superoxide dismutase (SOD).

### (Supplementary Description 13)

The test reagent according to Supplementary Description 12, wherein the measurement reagent contains a probe for detecting xanthine, xanthine oxidase, and superoxide.

### (Supplementary Description 14)

The test reagent according to Supplementary Description 12 or 13 for use in the test method according to any one of Supplementary Descriptions 4 to 11.

### (Supplementary Description 15)

A method for screening therapeutic candidate substances for mild cognitive impairment (MCI), the method including a step of selecting, as a therapeutic candidate substance for MCI, an activating substance that enhances activity of superoxide dismutase (SOD) from test substances.

### (Supplementary Description 16)

The screening method according to Supplementary Description 15, the screening method including a step of causing each of the test substances to be also present in a mixture containing superoxide and superoxide dismutase (SOD), and measuring the SOD activity; and

a step of choosing the test substance as the therapeutic candidate substance when the SOD activity obtained in the step of causing each of the test substances to be also present in a mixture containing superoxide and superoxide dismutase (SOD), and measuring the SOD activity is higher than that in a control mixture in which the test substance is not present.

### (Supplementary Description 17)

The screening method according to Supplementary Description 15 or 16, wherein the test substance is at least one selected from the group consisting of a low-molecular-weight compound, a peptide, a protein, and a nucleic acid.

### (Supplementary Description 18)

A method for detecting activity of superoxide dismutase (SOD) in a subject who has a suspected case of mild cognitive impairment (MCI), the method including:

a step of detecting SOD activity in a biological sample of the subject using a reagent for measuring the SOD activity.

### (Supplementary Description 19)

The detection method according to Supplementary Description 18, wherein the biological sample is saliva.

### (Supplementary Description 20)

Use of a reagent for measuring SOD activity in order to test the likelihood of mild cognitive impairment (MCI).

### INDUSTRIAL APPLICABILITY

As described above, according to the present invention, the likelihood of subjects having mild cognitive impairment (MCI) (morbidity risk) and the like can be tested by measuring the activity of superoxide dismutase (SOD). Also, in the present invention, because the activity of SOD serves as a target with respect to MCI, therapeutic candidate substances for MCI can be obtained through screening using the target. Therefore, the present invention is extremely useful in the clinical and biochemical fields.

1. A test method for mild cognitive impairment (MCI), the method comprising a step of measuring activity of superoxide dismutase (SOD) in a biological sample of a subject.

2. The test method according to claim 1, wherein an SOD activity value is determined in the step of measuring activity of superoxide dismutase (SOD) in a biological sample of a subject.

3. The test method according to claim 2, further comprising a step of testing the likelihood of the subject having MCI by comparing the SOD activity value in the biological sample of the subject with a reference value,
wherein the reference value is an SOD activity value in a biological sample of a healthy subject or an SOD activity value in a biological sample of an MCI patient.

4. The test method according to claim 3, wherein, in the step of testing, when the SOD activity value in the biological sample of the subject is lower than the SOD activity value in the biological sample of the healthy subject, when the SOD activity value in the biological sample of the subject is the same as the SOD activity value in the biological sample of the MCI patient, or when the SOD activity value in the biological sample of the subject is lower than the SOD activity value in the biological sample of the MCI patient, the subject is assessed as likely to have MCI.

5. The test method according to claim 1, wherein the SOD activity is superoxide scavenging activity.

6. The test method according to claim 1, wherein the biological sample is saliva.

7. The test method according to claim 1, wherein the testing for MCI is detection of MCI, determination of MCI, determination of an MCI preventive effect, determination of an MCI therapeutic effect, screening for MCI, determination of an MCI patient who responds to a therapeutic agent, determination of a therapeutic agent that takes an effect on an individual MCI patient, a test method for diagnosing MCI, or a test for treating MCI.

8. A test reagent for MCI, comprising a reagent for measuring activity of SOD.

9. The test reagent according to claim 8, wherein the measurement reagent contains a probe for detecting xanthine, xanthine oxidase, and superoxide.

10. The test reagent according to claim 8 for use in a test method for mild cognitive impairment (MCI) comprising measuring activity of superoxide dismutase (SOD) in a biological sample of a subject.

11. A method for screening therapeutic candidate substances for MCI, the method comprising a step of selecting, as a therapeutic candidate substance for MCI, an activating substance that enhances activity of SOD from test substances.

12. The screening method according to claim 11, the screening method comprising:
a step of causing each of the test substances to be also present in a mixture containing superoxide and SOD, and measuring the SOD activity; and
a step of choosing the test substance as the therapeutic candidate substance when the SOD activity obtained in the step of causing each of the test substances to be also present in a mixture containing superoxide and SOD, and measuring the SOD activity is higher than that in a control mixture in which the test substance is not present.

13. The screening method according to claim 11, wherein the test substance is at least one selected from the group consisting of a low-molecular-weight compound, a peptide, a protein, and a nucleic acid.

14. A method for detecting activity of SOD in a subject who is a suspected case of MCI, the method comprising:
a step of detecting SOD activity in a biological sample of the subject using a reagent for measuring the SOD activity.

15. The detection method according to claim 14, wherein the biological sample is saliva.
